(19) United States
(12) Patent Application Publication
(10) Pub. No.: US 2019/0094112 A1
(43) Pub. Date: Mar. 28, 2019

(54) BIOLOGICAL SAMPLER, COLLECTOR AND STORAGE CONTAINER

(71) Applicant: SNPSHOT TRUSTEE LIMITED, Auckland (NZ)

(72) Inventors: Michael Stuart GARDNER, Remuera, Auckland (NZ); Roy Victor BLADEN, Albany, Auckland (NZ); Rory BLADEN, Auckland (NZ)

(21) Appl. No.: 16/083,306

(22) PCT Filed: Feb. 27, 2017
(86) PCT No.: PCT/IB2017/051119

§ 371 Date: Sep. 7, 2018

Related U.S. Application Data
(60) Provisional application No. 62/316,499, filed on Mar. 31, 2016.
(60) Provisional application No. 62/305,498, filed on Mar. 8, 2016.

(30) Foreign Application Priority Data
Mar. 31, 2016 (NZ) 718587
Nov. 2, 2016 (NZ) 725835
Nov. 2, 2016 (NZ) 725837

(51) Int. Cl. G01N 1/28; A61B 10/02

(52) U.S. Cl. CPC G01N 1/286; A01K 11/003; G01N 2001/288; A61B 10/0266

(57) ABSTRACT

The invention relates to, as an assembled elongate string, a sample collector and sample collector driver to drive at least the sample collector through an item to be sampled. The sample collector comprises a punch having a bore aligned to the elongate direction. One end of the punch and bore presents a cutter to cut and hold a sample from the item at the bore as the punch passes through the sample. The sample collector further comprises a plunger slideably supported to said punch to be able to move in said bore between a retracted position and an advanced position relative said punch. The movement of the plunger from the retracted position to the advanced position causes, in use, the cutter held sample to be displaced from the cutter. The sample collector driver comprises a body interfaced with said sample collector in a telescopic manner.

### FIELD OF THE INVENTION

The present invention relates to improvements in or relating to biological sample collecting.

### BACKGROUND

The collection of biological samples such as animal tissue, typically collected from the ear of an animal needs to be done efficiently and without risk of contamination of the sample collected from prior samples that have been collected. As an example, large herds of cows may from time to time need to be sampled at a given time. If a sampler tool and sample collectors are to be individually handled for each cow, the process can become time consuming. There is also a risk that cross contamination of samples may occur. In addition, in order to ensure that a sample taken from an animal is not tampered with, it is important to have sufficient deterrents in place to prevent sample tampering yet at the same time ensure that matching with other animal information and/or tracking of the sample, usually retained in a sealed storage container, is possible.

Where used in this specification tissue means any part of a living thing, particularly any part made up of similar cells, or any part or parts that perform a similar function. Tissue preferably refers to any form of biological sample, from plants and animals particularly, including pigs, goats, cattle, sheep, poultry, and fish. Biological samples may include for example, animal tissue such as flesh, blood, hair, fur, saliva, sweat, urine, etc, or plant tissue such as leaves, bark, roots or wood, or any other part of a plant or animal but particularly those that are made up of similar cells, or which perform a similar function.

The present invention may be used at least for either or both of production animals and companion animals. It is anticipated that production animals may include but not be limited to bovine, pigs, deer and sheep. Further it is anticipated that companion animals may include but not be limited to horses, cats and dogs.

### BRIEF DESCRIPTION OF THE INVENTION

Accordingly the present invention may be broadly be said to be, as an assembled elongate string, a sample collector and sample collector driver to drive at least the sample collector through an item to be sampled along a sampling axis aligned to the elongate direction of the string,
the sample collector comprising:
a punch presenting at one end a cutter to cut and hold a sample from the item as it passes there through, the punch having a bore aligned to the elongate direction,
a plunger slideably supported at said bore to be able to move between a retracted position and an advanced position relative said punch, the movement from the retracted position to the advanced position causing, in use, the cutter held sample to be displaced from the cutter,
the sample collector driver comprising
a body interfaced with said sample collector in a telescopic manner wherein in a telescopically compact position the driver can drive the sample collector through the item whilst keeping the plunger in the retracted position so that a sample can be cut and held by the cutter and in a telescopically expanded position the driver is able to be driven in the same direction as when driving the collector through the item and cause the plunger to move from its retracted position to its advanced position.

Preferably when the sample collector and the body are in the telescopically compact position the body and the punch are in contact with each other so that the body, when being driven, pushes onto the punch to drive the sample collector through the item.

Preferably when the sample collector and the body are in the telescopically compact position the body and the punch are in contact with each other so that the body, when being driven, pushes onto the punch and not onto the plunger, to drive the sample collector through the item.

Preferably when the sample collector and the body are in the telescopically compact position the body and the punch are in contact with each other so that the body, when being driven, pushes onto the punch and does not cause the plunger to move to its advanced position, to drive the sample collector through the item.

Preferably when the sample collector and the body are in the telescopically expanded position the body and the plunger are in contact with each other so that the body, when being driven in the same direction, pushes onto the plunger to move the plunger to its advanced position.

Preferably when the sample collector and the body are in the telescopically expanded position the body and the plunger are in contact with each other so that the body, when being driven in the same direction, pushes onto the plunger and not onto the punch, to move the plunger to its advanced position.

Preferably when the sample collector and the body are in the telescopically expanded position the body and the plunger are in contact with each other so that the body, when being driven in the same direction, pushes onto the plunger and does not cause the punch to move, to move the plunger to its advanced position.

Preferably one of the body and the plunger carries a dog that in said compact position is help in an inactive mode preventing the driving of the plunger by the body to its advanced position and in the expanded position couples said plunger and body.

Preferably the dog is a resiliently flexible member of one of said body and plunger, held under bias when in its inactive mode by the other of said plunger and body when the sample collector and the body are in the compact position.

Preferably the dog is a resiliently flexible member of one of said body and plunger, biased towards a position that, when the sample collector and the body are in the expanded position, presents itself for engagement with a driving surface of the other of said plunger and body so that when said body is moved in the same direction, the plunger is driven towards its advanced position.

Preferably the dog is carried by said plunger and is able to align to index with a said driving surface of said body when the body is in the expanded position relative said sample collectors.

Preferably the sample collector is generally of circular cross section and elongate and straight, the cutter located at one end of the collector.

Preferably the punch is generally of circular cross section and elongate and straight, the cutter located at one end of the collector.

Preferably the punch is generally of circular cross section and elongate and straight, the cutter located at one end of the collector.

Preferably the plunger protrudes from the end of the punch opposite the cutter.

Preferably the plunger includes an umbrella acting as said dog, the umbrella resiliently moveable between a contracted condition and an expanded condition wherein the umbrella is (a) held in one of said contracted and expanded conditions by a lateral surface or surfaces of said body along which said umbrella can slide when the body moves to the expanded position relative the sample collector and (b) can move to the other of the expanded or contracted conditions when the body has moved to the expanded position so that the umbrella can engage said body to allow the body to move the plunger to the advanced position.

Preferably the plunger is rod shaped.

Preferably umbrella is located at an end of the plunger distal from most the cutter.

Preferably the body is elongate and straight.

Preferably the body is elongate and straight and of the same exterior cross section as the punch.

Preferably the body and the punch form a continuous elongate drivable member when in the compact position.

Preferably the body includes a receptacle region to receive a driver, directly or indirectly, of a sampler device that can apply a force to cause the sample collector to be driven through the item.

Preferably the receptacle region is adapted and configured so that it can be engaged by said driver, directly or indirectly, to cause the body to be displaced in a driving direction and in a direction opposite the driving direction.

Preferably the body is to seal the entrance of a sample storage container with which the string is to engage after the sample has been taken.

In a further aspect the present invention may broadly be said to be, as a set a string as herein described and a sample storage container defining a mouth opening leading to a storage region of the storage container, the mouth opening able to receive the string after the cutter has been driven through the item and is carrying a sample.

Preferably the storage container is adapted and configured to retain the sample collector (preferably the punch) once received at said mouth opening in a manner to allow the driver to be moved relative said sample collector and said storage container in a direction to move it to the expanded position relative the sample collector.

Preferably said body advances back towards punch as it drives said plunger to its advanced position, the so advancing body causing a sealing of said mouth opening.

Preferably said mouth opening and said body include a region of mutually cooperative surface or surfaces that allow the body to seal the mouth opening and hence the containment region of the storage container.

Preferably container comprises a container body and a cap removably engaged to the container body, the mouth opening is a passage defined by said cap.

Preferably said passage and said body of the driver are adapted and configured to engage in a snap-fit manner to retain the body of the driver with the cap and seal the containment region.

In yet a further aspect the present invention may broadly be said to be a method of taking and storing a sample from an item using the set as herein described, by
placing the storage container and string on opposed sides of a gap, held by a sampler device in axial alignment, the sampler device including an actuator to act, directly or indirectly on said sample collector driver,
placing a part of an item to be sampled in said gap so that part of the item is also in axial alignment,
causing the actuator to move in a driving direction to drive said string towards said storage container so that said sample collector is driven into the mouth opening of the storage container so that said punch becomes held by said storage container,
causing the actuator to move in a direction opposite said driving direction to allow the sample collector driver to register with said plunger for the purposes of driving said plunger in said driving direction, and
causing the actuator to move in the driving direction to cause the sample collector driver to displace the plunger to its advanced position.

Preferably at step (a) and (b) and (c) the plunger is in its retracted position relative said punch and said sample collector driver is in the compact position relative said punch.

Preferably at step (d) the plunger is in its retracted position relative said punch and said sample collector driver is in the expanded position relative said punch.

Preferably at step (e) the plunger is in its advanced position relative said punch and said sample collector driver is in or more proximate its compact position relative said punch than its said expanded position.

Preferably at step (e) the sample collector driver is driven into the mouth opening in a manner to seal the mouth opening.

In still a further aspect the present invention may broadly be said to be a sampling assembly to be driven in one direction relative to and to collect and dispense a biological sample from an item and into a storage container, said assembly comprising:
a sample collector comprising:
a punch presenting at one end a cutter to cut and hold a sample from the item as it passes there through or over,
a plunger slideably supported by said punch to be able to move between a retracted position and an advanced position relative said punch, movement from the retracted position to the advanced position causing, in use, the cutter held sample to be displaced from the cutter,
a sample collector driver comprising:
a body telescopically interfaced with said sample collector to, in a first position relative said punch and plunger, be able to drive said punch and plunger in said one direction, and in a second position relative said punch and plunger, having moved in a direction opposite said one direction, having ratcheted to engage with said plunger in a manner to be able to cause said plunger to move relative to said punch when said sample collectors driver is again driven in said one direction.

In still a further aspect the present invention may broadly be said to be a sampling string to collect a biological sample from an item, said string comprising:
a sample collector comprising:
a punch presenting at one end a cutter to cut and hold a sample from the item as it passes there through or over,
a plunger slideably supported by said punch to be able to move between a retracted position and an advanced position relative said punch, movement from the retracted position to the advanced position causing, in use, the cutter held sample to be displaced from the cutter,
a sample collector driver that in a first condition is engaged said punch to, in use, be able to drive said punch over or through said item when in a first position relative punch and is able to telescopically slide relative said plunger to a second condition that is away from and/or not engaged to said punch to ratchet with said plunger and engage therewith to be able to drive the plunger from its retracted position to its advanced position.

Preferably in said first condition the driver is prevented from driving the plunger to its advanced position.

Preferably in said first condition the plunger is not in driving contact with said driver and in said second condition the punch is not in driving contact with said driver.

Preferably in said first condition the plunger is not in driving contact with said driver and in said second condition the punch is not in driving contact with said driver.

Preferably said string is an elongate straight string and when said driver is in said second condition said string is longer than when in said first condition.

Preferably said driver includes a recess into which the plunger of the sample collector extends at least when in the first condition.

Preferably said plunger projects from said bore and into driver.

In still a further aspect the present invention may broadly be said to be a sample collector to be driven by a driver to remove and hold a sample from the item that it can be driven over/through comprising:
a punch presenting a cutter and presenting a driver engageable region to allow the driver to drive said punch to remove said sample from said item,
a plunger slideably supported by said punch to be able to move relative said punch between a retracted position and an advanced position, movement from the retracted position to the advanced position causing, in use, the cutter held sample to be displaced from the cutter, the plunger adapted and configured to ratchet with said driver in moving from a first position relative said driver to a second position relative said driver, such that when in said first position said driver not move the plunger from its retracted position to its advanced position and in said second position said plunger is coupled to said driver to allow the driver to move the plunger to its advanced position.

In still a further aspect the present invention may broadly be said to be a sampling string to collect a biological sample from an item, said string comprising:
a sample collector comprising:
a punch presenting at one end a cutter to cut and hold a sample from the item as it passes there through or over,
a plunger slideably supported by said punch to be able to move between a retracted position and an advanced position relative said punch,
a sample collector driver that in a first condition is engaged said punch to, in use, be able to drive said punch over or through said item,
wherein movement from the retracted position to the advanced position causing, in use, the cutter held sample to be displaced from the cutter, and actuated by a telescopic displacement of the driver from the punch that ratchets the plunger and driver into an operative driving condition.

In yet a further aspect the present invention may be broadly be said to be a cartridge comprising a body that include a storage container holding region at where a storage container as herein described is located and a sample collector holding region at where a sample collector as herein described is located, preferably with the sample collector driver engaged with said sample collector in said condition where said plunger is in its retracted position and unable to be moved by said sample collectors driver to its advanced position, there the body holds the sample collector and storage container in alignment with each other for sample taking and spaced apart with a sufficient gap to allow part of an item to be sampled to locate at said gap.

Preferably the cartridge is adapted and configured to be loadable and unloadable to/from a sampler device that includes and actuator to cause sampling to take occur.

Preferably the body of the cartridge is adapted and configured to shield the sampler device at the gap from said item placed in the gap, and any exudates expressed or expelled from said item during sampling.

Preferably one (or more) of the invention(s) herein described may be used for production animals and for companion animals.

Preferably one (or more) of the invention(s) herein described may only be used for production animals.

Preferably one (or more) of the invention(s) herein described may only be used for companion animals.

Preferably production animals include but are not limited to bovine, pigs, deer and sheep.

Preferably companion animals include but are not limited to horses, cats and dogs.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The invention will now be described by way of example only and with reference to the drawings in which:

FIG. 1 shows an exploded view of a storage container axially aligned with a sample collector and sample collector driver plus an associated push rod,

FIG. 2 is an exploded view of the string of components comprising of the sample collector comprising of the punch and plunger and the sample collector driver and push rod,

FIG. 3a is a cross sectional view through the components of FIG. 2 showing the plunger with its umbrella in an expanded, drivable condition,

FIG. 3b shows the umbrella of the plunger in a contracted condition,

FIG. 4 is a perspective view of the storage container and sample collector/sample collector driver/push rod string intermediate of which part of an ear of an animal is placed prior to sampling,

FIG. 5 is a cross sectional view of the components of FIG. 4 with the cutter of the sample collector having punctured the ear of the animal,

FIG. 6 is a cross sectional view of the components of FIG. 4 wherein the sample that has been collected from the ear of the animal is driven into the storage container,

FIG. 7 shows the components of FIG. 4 and wherein the punch is about to rupture the frangible seal of the cap of the storage container,

FIG. 8 is a view of the components of FIG. 4 wherein the frangible seal has been ruptured to allow for the sampler to enter the storage region of the storage container,

FIG. 9 shows the step in the process of the components of FIG. 4 wherein the sample collector driver has retreated back towards its pre-delivery position in order to cause a telescopic movement relative to the plunger and allow for the two components to ratchet to a second condition so that the plunger is then able to be actuated to move to an advanced position by the sample collector,

FIG. 10 is a cross sectional view of the sample collector driver and the plunger, the plunger and its umbrella shown in a contracted condition,

FIG. 11a is a view of FIG. 10 but wherein the umbrella of the plunger is shown in unexpanded condition,

FIGS. 11b-j show variations of the telescopic ratchet relationship that can be established between the sample collector driver and the plunger in order to allow for the plunger and the sample collector driver to move between a retracted and advanced position and where a ratcheting relationship is provided by varying features of the sample collector driver and the punch,

FIG. 12 shows the sample collector driver and plunger in a telescopically expanded relationship, the plunger still being in a retracted condition relative to the punch but in a position able to be driven by the sample collector driver to an advanced position,

FIG. 13 illustrates the sample collector driver and plunger indicating that contact may exist between the interior wall of the sample collector driver and the driving end of the plunger,

FIG. 14 shows the sample collector driver and plunger wherein the plungers umbrella is in an expanded condition able to then be driven by a different surface of the sample collector driver,

FIG. 15 illustrates the sample collector driver having been advanced, after the momentary retraction, back towards the storage containment region in order to then drive the plunger relative to the punch to cause the sample to drop into the storage containment region and wherein the sample collector driver has engaged at the passage of the cap of the storage container in order to seal the storage container,

FIG. 16 shows the push rod having been retracted from the sample collector driver and the sampling and storage process being complete,

FIG. 17 is an enlarged view of the string of components including the sample collector, the sample collector driver and the push rod wherein these components are in a condition for punching through the ear of an animal,

FIG. 18 shows the components of FIG. 17 but wherein the sample collector driver has telescopically moved relative to the sample collector and to its expanded position relative thereto in order to ratchet with the plunger for the purposes of then re-advancing for the purposes of driving a plunger relative to the punch as can be seen in FIG. 19,

FIG. 19 shows the plunger having been moved to its advanced position relative to the punch by a re advancement of the sample collector driver,

FIG. 20 shows an assembly of the storage container and the string of components including the sample collector and sample collector driver associated with a cartridge, the cartridge being able to be delivered to an end user with the sample collector related string and storage container in pre alignment with the gap between at where the ear of an animal may be placed,

FIG. 21 is a cross sectional perspective view of FIG. 20,

FIG. 22 shows a partially expanded view of the components of FIG. 20,

FIG. 23 shows a cross sectional view of the components of FIG. 20,

FIG. 24 shows a cross sectional view of the components of FIG. 20 but wherein the string of components including the sample collector is advanced towards the storage container immediately prior to the taking of a sample from the ear of an animal,

FIG. 25 shows the storage container having received the sample and the sample collector having sealed the storage container,

FIG. 26 shows a perspective view of a sampler device with the cartridge of FIG. 20 loaded therewith,

FIG. 27 is another perspective view of FIG. 26,

FIG. 28 is a side view of a sample collector device and the cartridge in an expanded condition,

FIG. 29 is a perspective view of FIG. 28,

FIG. 30 is a cross sectional view of the sampler device and the cartridge loaded therewith including showing part of the mechanism for the purposes of driving the string of components including the sample collector through the ear of an animal and wherein the mechanism as shown in a condition open prior to the taking of a sample,

FIG. 31 shows the mechanism of FIG. 30 and wherein the sample collector string has been advanced towards the storage container, the sample collector having entered the storage container and the frangible membrane having been ruptured but prior to the mechanism of the sampler device having been actuated to cause the plunger to move to its advanced position,

FIG. 32 is a view of the mechanism of the sampler device and wherein the mechanism is in a condition to advance the plunger relative to the punch for the purposes of dispensing the sample into the sample containment region, and

FIG. 33 is a view of the mechanism of the sampler device wherein the actuator of the sampler has driven the sample collector driver to its final position to seal the storage container and to move the plunger to its advanced position to dispense the sample into the storage containment region.

FIG. 34 shows a view of the mechanism of FIG. 30 with further components according to one embodiment for an operation of the drive rod.

### DETAILED DESCRIPTION OF THE INVENTION

Reference will now be made to various components and variations of such components that can be used for the purposes of collecting and storing biological samples such as tissue samples from animals. Reference will first be made to the collection and storage components whereafter reference will be made to a sampler device and related components that can be utilised to take the samples using the collection and storage components.

Biological samples may be taken by the sample collectors herein described from an organism such as a plant or an animal particularly including pigs, goats, cattle, sheep, poultry and fish. In a preferred form the sample is a tissue sample that is taken from the ear of an animal.

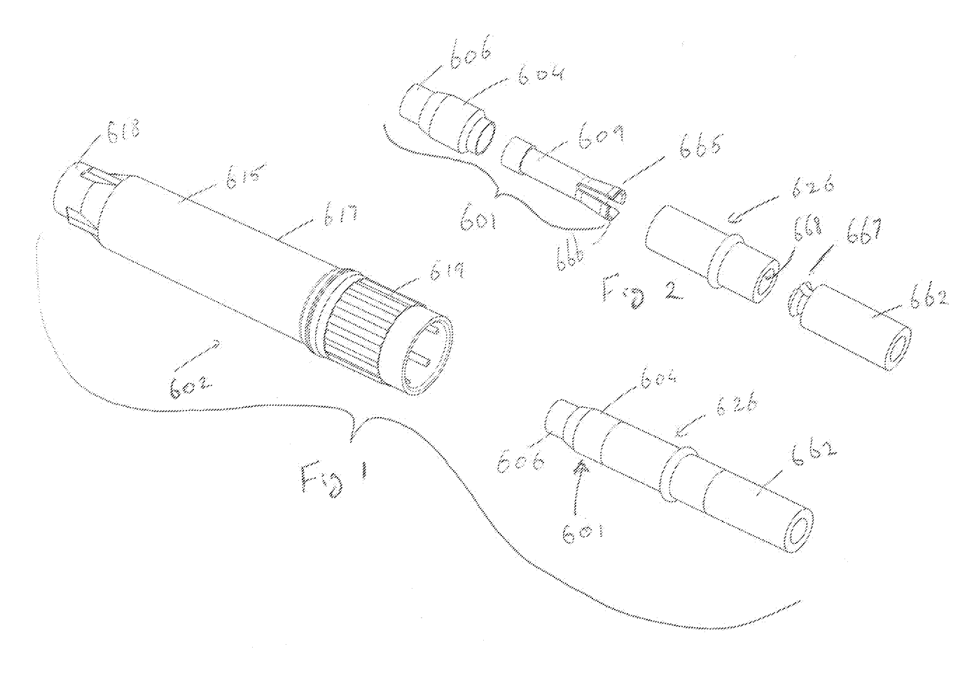

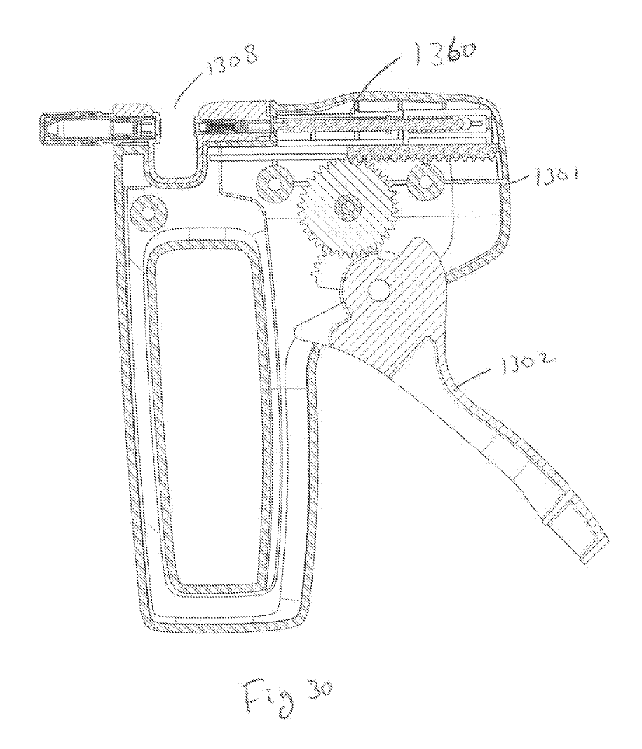

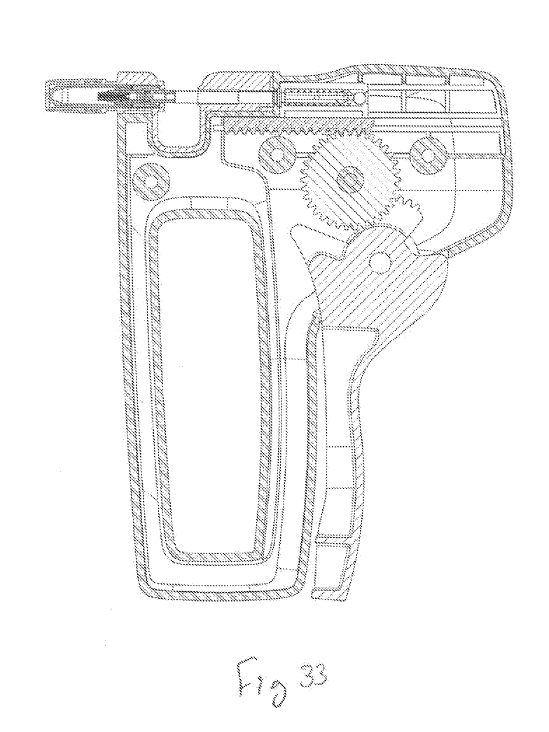

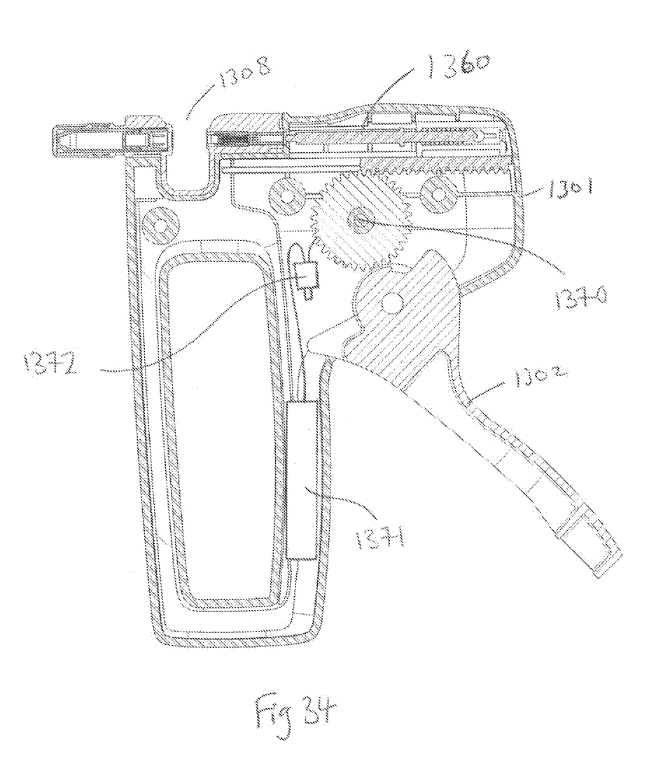

With reference to FIGS. 1 to 33 a sample collector 601 and storage container 602 is shown. The collector is able to be driven by a tool such as the tool shown in the drawings to engage (and preferably become secured) with the container that is also held by the tool in a predetermined position aligning the two together.

As seen in FIG. 1 a sample storage container 602 is shown that includes a container body 615 that has a containment region 616 that is terminated by an end wall 618 and a side wall 617 and includes a cap 619.

In the preferred form the end wall 618 of the containment region 616 is closed and not openable. It may alternatively be openable. In the preferred form the side and end walls are integrally formed. The container body 615 is preferably made from a plastics material. It may be a moulded plastics material. A preservative may be provided inside of the containment region 616.

The storage container 602 can receive a sample collector retained sample. Prior to receiving a sample the containment region 616 is preferably sealed. In the preferred form the storage container 602 also includes an end at where the sample collector penetrates the storage container, preferably in the form of a cap 619 that is either integrally formed with or secured and preferably removably secured to the container body 615. The cap 619 receives the sample collector 601 upon the taking of a sample such as from an animal's ear.

The cap 619 is preferably threadingly engaged to the container body 615 by virtue of threads 620. The cap can be screwed onto and preferably partially ascend into the container body 615. It can be unscrewed from the container body to gain access to the containment region. This may occur in a laboratory to gain access to the sample in the containment region.

In the preferred form the cap has a passage 621 that has an entrance 622 and an end 623 opposed at the entrance 22. The passage 621 is of a shape and configuration to be able to snugly receive the sample collector 601. It is preferably cylindrical in shape. In the preferred form the side walls of the passage 621 correspond substantially to an exterior side wall of the punch 604. Such an exterior side wall is preferably round in cross sectional shape. Preferably no gap or passage exists between the cap and the sample collector when the sample collector is located in the passage. This helps seal the storage region 616.

Within the passage and preferably at the end 623 opposed the entrance, the passage is sealed by a frangible seal 624. This could also be a plug or a membrane. The frangible seal 624, when the cap 619 is secured to the container body 615 and prior to the sample collector being received, seals the containment region 616 of the container body. The frangible seal 624 is preferably frangeably attached so that when a sample collector is delivered into the passage 621 it is able to push against the seal 624 to at least partially separate the seal 624 and allow for the sample to be pushed into the storage region 16. In the preferred form the sample is still retained in the bore 608 at the cutting end of the punch 604 when the frangible seal is at least partially separated to open the storage region 616.

The cap preferably has an additional function and/or feature to help seal the containment region, that will herein be described.

The sample storage container 602 is able to receive the sample collector 601 also shown in FIG. 1.

The sample collector 601 includes at a cutting end 606, a cutter 605 associated with or part of a punch 604.

The sample collector 601 shown in FIG. 4 is in a pre-sampling condition relative to the organism 603 to be sampled and the storage container 2, aligned with the storage container 602 intermediate of which for example a part of an animal's ear 603 is positioned.

The sample collector 601 comprises a punch 604 having a body with a cutter 605 presenting a cutting end 606 that is able to penetrate into the organism to be sampled.

The cutter 605 is provided at a first end of the punch 604. The punch has an opposing driving end 607. It is at the driving end that the punch can for example be driven by a driver that will hereinafter be described for the purposes of pushing the cutting end (and preferably the entire collector) at least partially into and preferably all the way through the organism to be sampled to take a sample. In the preferred form the entire sample collector 601 is pushed through the tissue to be sampled.

The body of the punch 604 preferably has a bore 608. The bore 608 extends from one end of the punch 604 to the other. It preferably extends along the length of the punch 604 between the cutting end 606 and the driving end 607. Preferably the punch 604 is an elongate straight body and the bore is centrally located within the punch. The cutter 605 defines at least part of this bore. The bore is preferably circular in cross section.

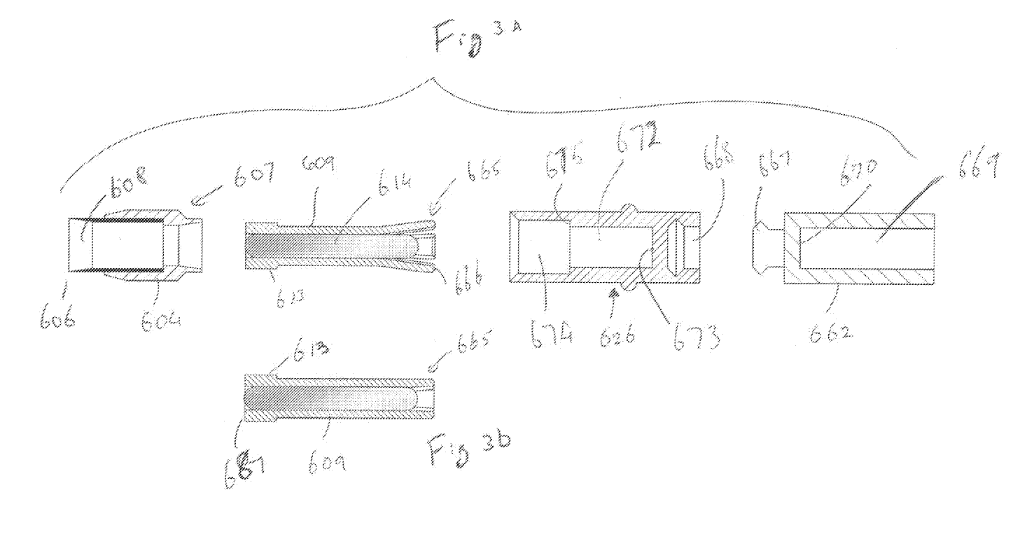

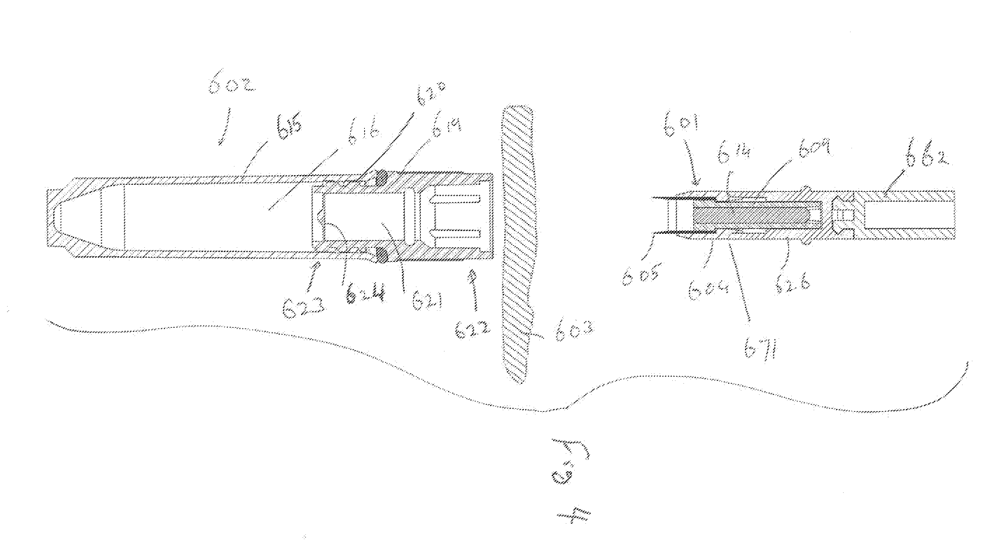

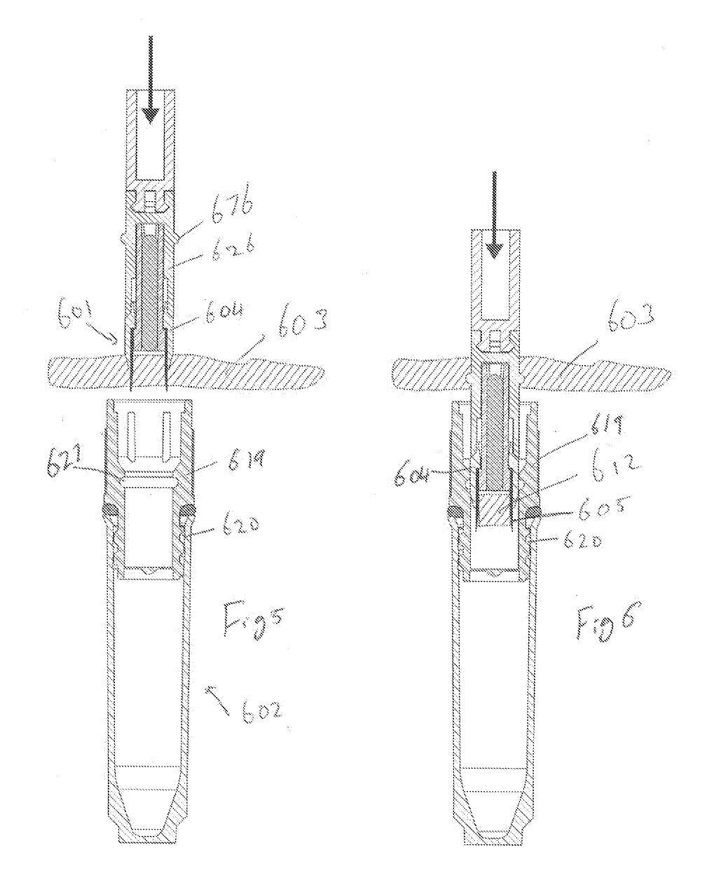

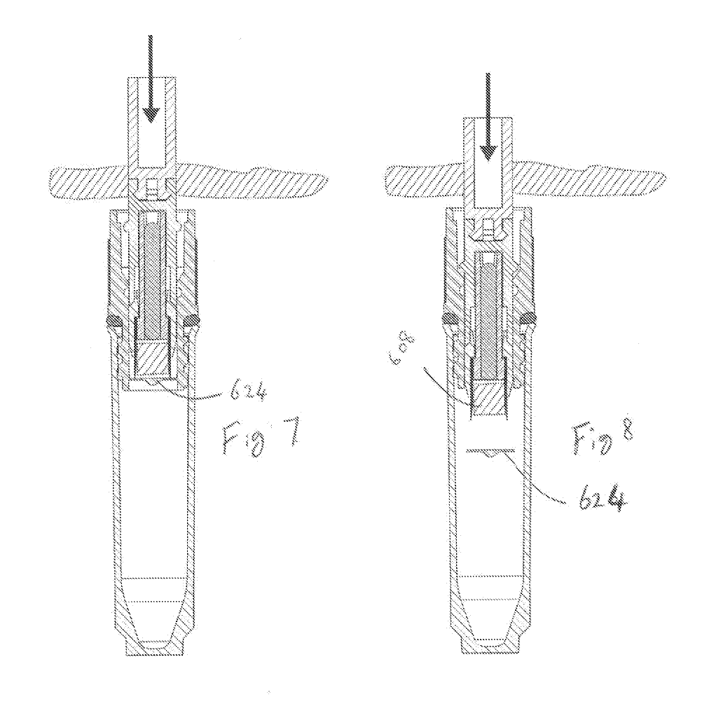

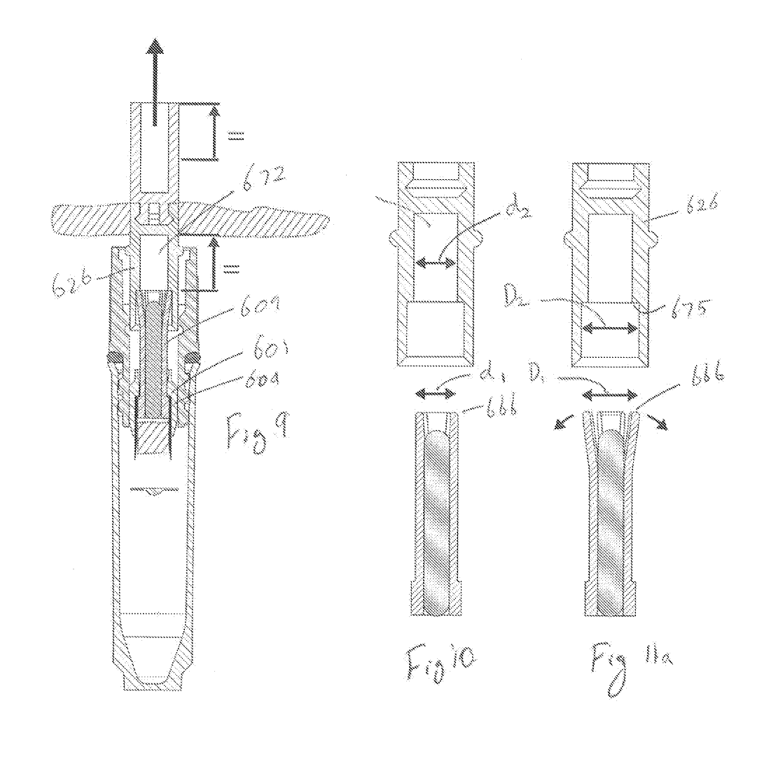

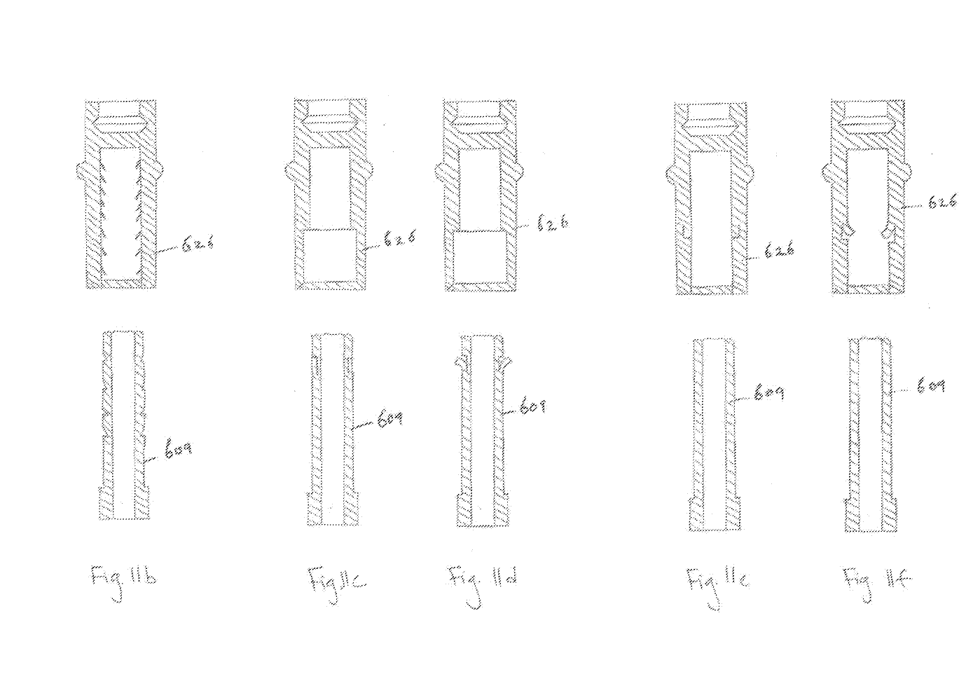

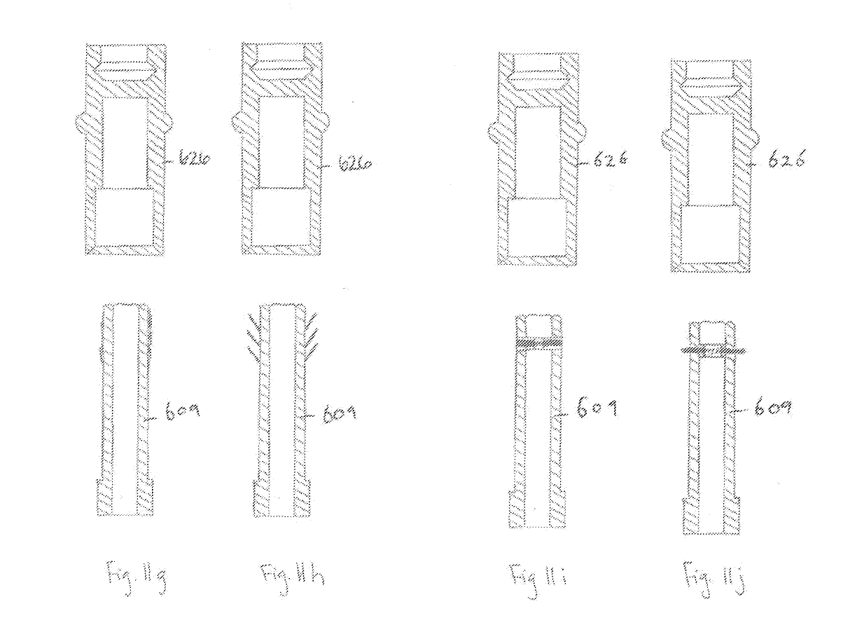

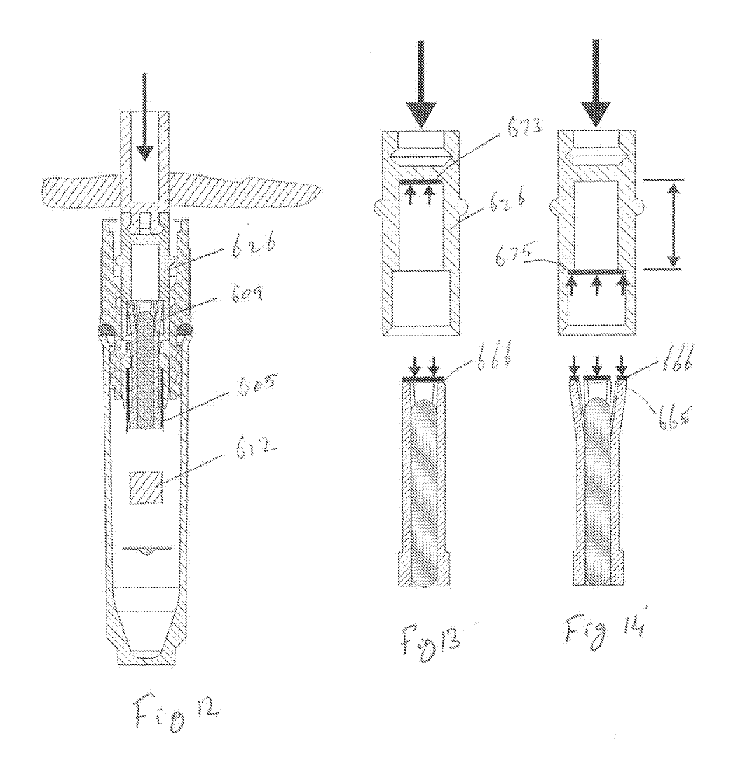

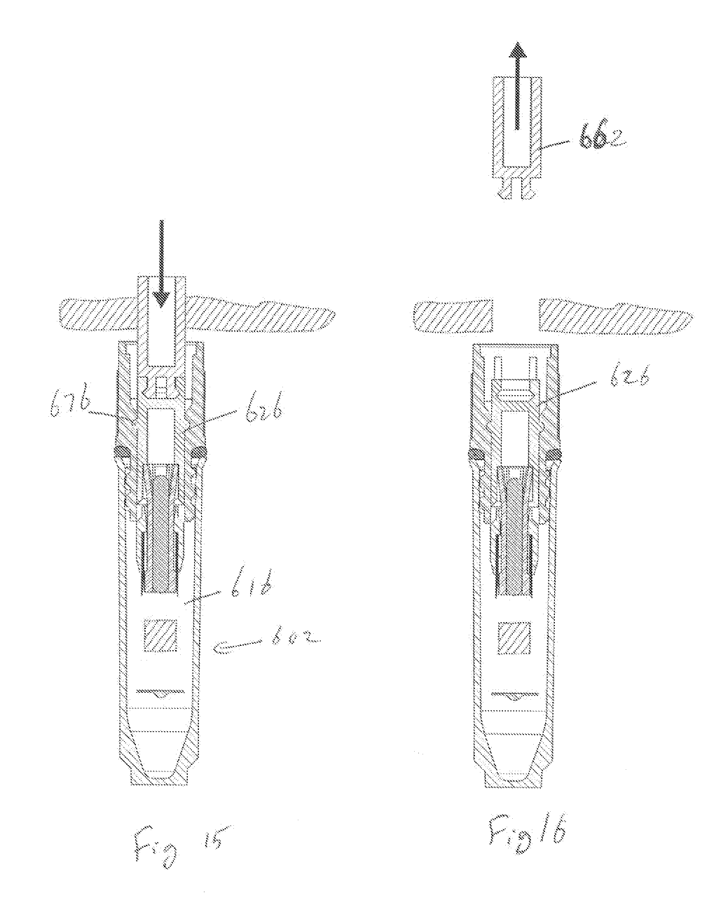

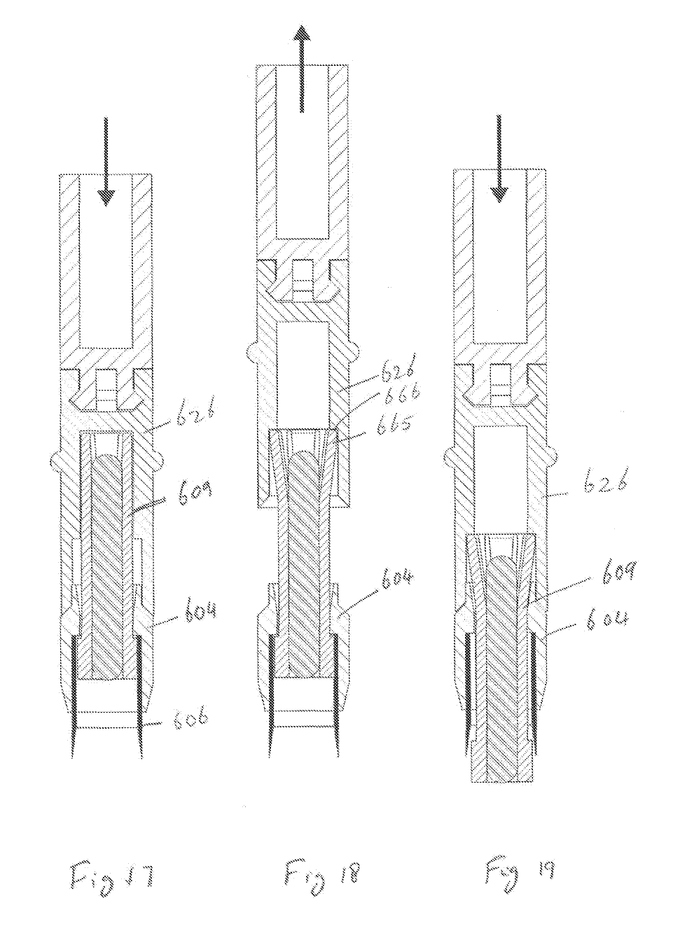

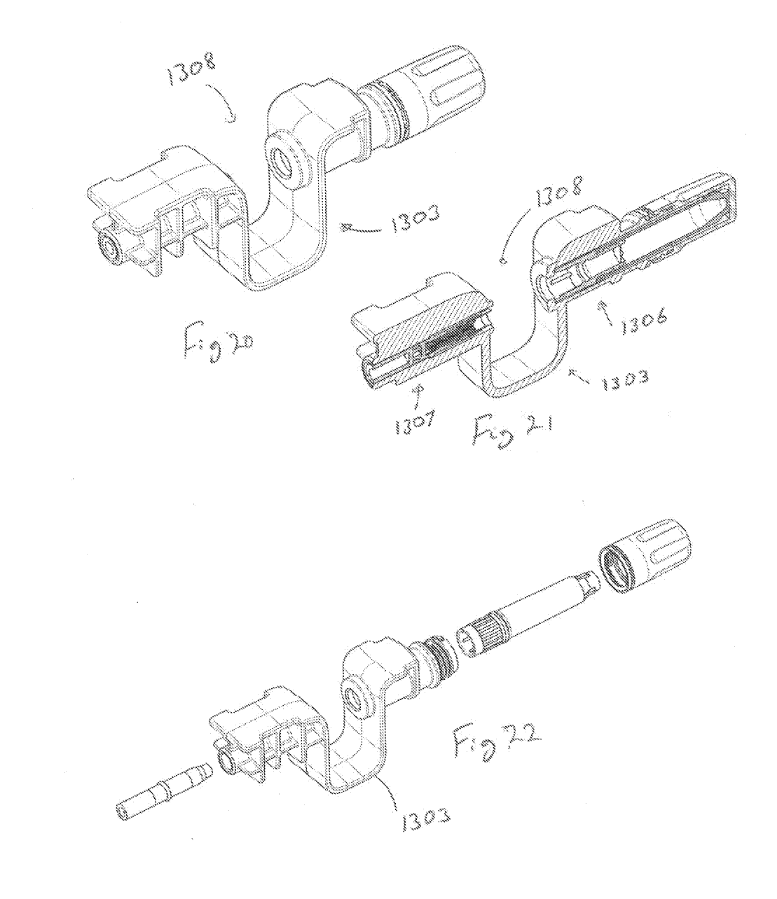

In the preferred form as can be seen in FIG. 2, the cutter 605 preferably has a circular cutting end. Preferably the cutter is substantially cylindrical shaped. It will be appreciated that alternative shapes can be used. It may be 3 or more sided for example.

The cutter 605 is provided at the cutting end of the punch to facilitate removal of a sample from an organism. The cutter may be attached to the punch or it may be integral with the punch so that the cutter and the punch are formed as a single part. It need not be formed to take a core sample by pushing through the organism but a sample instead taken at an edge of surface of the organism. However, being of a hollow section such as a cylindrical section offers the added benefit of the cutter being able to retain the sample as a plug. When driven into and preferably through an organism the sample is cut from the organism and becomes retained at the bore 608 of the punch. The cutter 605 preferably extends from and surrounds one end of the bore 608 of the punch at the cutting end of the punch.

In the form shown in FIGS. 1 and 2, the bore 608 is effectively a blind bore by virtue of the provision of a plunger 609 being located in the bore of the punch 604. The plunger 609 is aligned with the bore. The plunger 609 is held at the bore 608 of the punch to form part of the sample collector 601. In one form the plunger protrudes at least partially from the bore as seen in FIG. 4. The plunger and the punch in a pre-sampling condition are configured so that a cavity, extending inwards from the cutting end 608 and that is part of the bore, is provided for a sample to be collected in as the sample collector 601 is driven into and/or through the organism.

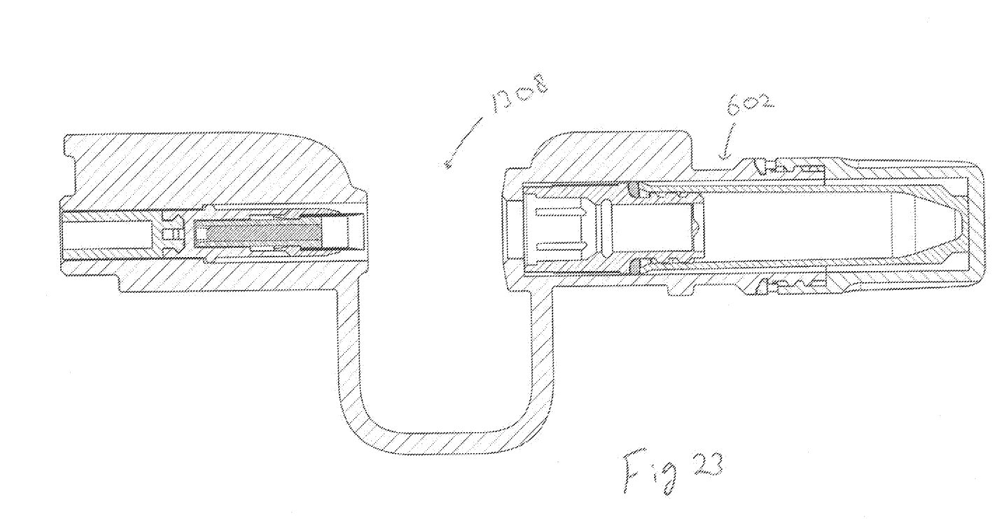

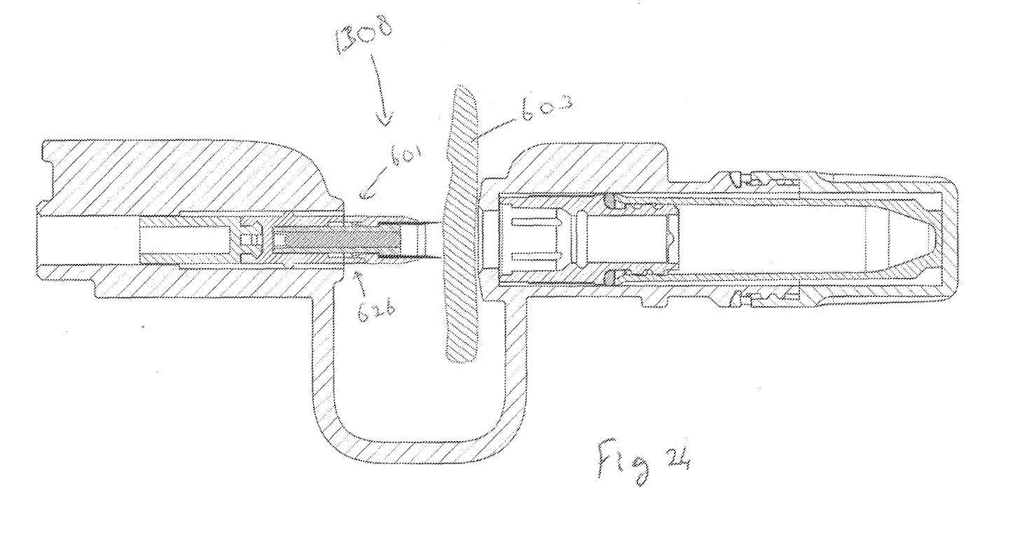

The plunger has a first end 687 and a second end 666. The first end is a pushing end to push onto the sample to push it out of the cavity. The second end is the driving end to drive the plunger in a manner that will herein after be described. The fit of the plunger 609 in the bore 608 is snug yet allowing for the plunger to be slid relative to the punch. In the preferred form the plunger's outer surface is contiguous the inner surface of the bore. This ensures that a close fitting configuration is provided between the punch 604 and the plunger 609 thereby helping to prevent the ingress of contaminants from the driving end of the punch to or towards the cutting end of the punch through the bore. The plunger and the punch are in a slidable relationship with each other. They are in a slidable relationship with each other so that a sample, once collected from the organism and retained in the bore at and near the cutting end 606 can be ejected therefrom by the plunger 609. Preferably the first end is able to be pushed all the way to the cutting end 606 to thereby eject the sample collected from the sample collector. Actuation of the plunger may be by pushing at the first end 10 of the plunger. The plunger is able to be positioned in an active position as shown in FIG. 17 and be moved to a plunged position as shown in FIG. 19 in a manner herein after described.

Preferably the plunger 609 includes an enlarged region 613 that prevents the plunger from being pulled out of the punch 604 from the driving end 607. The enlargement and a corresponding constriction in the bore of the punch limit the movement of the plunger and it cannot be removed by pulling the plunger out of the punch from the driving end 607. This helps prevent external access being gained to the sample once collected and it held at the cavity or dispensed further into the containment region 616 of the storage container.

In the preferred form the actuation of the plunger causes the sample to eject from the bore and into the containment region 616.

The plunger 609 preferably includes an EID tag 614.

The plunger as has hereinbefore been described, can move axially along the bore of the punch to allow for the sample that is collected by the cutter 605 to be pushed from the collector and into the containment region 616 of the storage container 602. The plunger includes a pushing end 687 that is able to push the sample from the cutter and it includes a driving end 666.

At the driving end opposite the cutting end, the sample collector 601 is preferably able to be pushed to be displaced by a sampler device through for example the ear of an animal in order to cause the sample to be collected.

The sample collector 601 is associated with a sample collector driver 626 during the process of sampling. The sample collector and sample collector driver may be assembled as an elongate string. In use, the sample collector driver first pushes the sample collector through the ear of an animal. It can then be caused to push the plunger to dispense the sample into a containment region and then optionally to seal the containment region. This will now be described.

The sample collector driver 626 is provided for sampling, connected to the sampler collector, as is for example seen in FIGS. 4 and 5. The form of the connected sample collector driver and sample collector is such that the components are axially aligned and resemble an elongate (preferably straight) string akin to a drill string, along the aligned axis.

In the preferred form a push rod 662 is removably connected to the sample collector driver 626 during the process of sampling. The push rod 662 is optionally provided for the purposes of driving the sample collector and the sample collector driver through the ear of an animal and provides an extension or a sleeve about the driving rod of the sampler device 1300. This helps to avoid cross contamination of samples during the sampling process. The push rod 662, once sampling has occurred, is able to be removed from the sample collector driver 626. As can be seen in FIG. 3a an interference fit plug 667 is provided. The interference fit plug 667 is able to locate in a socket 668 at an end of the sample collector driver 626 but is able to be removed therefrom upon the application of a sufficient force. The assembly of the sample collector 601 and the sample collector driver 626 and optionally where included the push rod 662 are substantially of a slender elongate configuration that is substantially of a constant diameter or other external profile. This helps ensures that the assembly is able to be pushed through the ear of an animal to collect a sample. As can be seen the sample collector and sample collector driver and push rod, are preferably elongate and straight and serially connected as in a drill string. They are preferably axially aligned.

The push rod 662 as seen in FIG. 3a is preferably of a configuration to act as a sleeve for the actuator driver of the sampler device. A cavity 669 is provided into which the actuator driver can locate to then drive the assembly of the sample collector, the sample collector driver and the push rod by pushing on the end wall 670 of the push rod 662.

The plunger 609 has a telescoping relationship with the sample collector driver 626 such that the plunger and the sample collector driver may move between a first and second position relative to each other. Facilitated by the telescoping relationship the plunger and sample collector driver may be engaged in a ratchet-like connection. The ratchet-like connection acts to allow movement in one telescoping direction but then to prevent motion or to prevent motion past a certain point in the opposite direction. The ratchet-like connection may be in the form of a dog or umbrella like formation 665 or other physical obstruction or engagement provided by either the plunger or sample collector driver or by provided an interaction of both the plunger and sample collector driver.

Several examples of possible ratchet-like connections are shown in FIGS. 11b-j. In the preferred embodiment the plunger may include a driving end 666 that is defined by an umbrella 665. The umbrella is naturally biased to an expanded condition as shown in FIG. 14 but can be caused/forced to assume a contracted condition as seen in FIG. 13.

The relationship between the sample collector driver 626 and the punch 604 and the plunger 609 is such that the three components are able to axially displace relative to each other. This will now be described. The sample collector and sample collector driver and storage container are able, for the purposes of sampling, to be held by a sampler device 1300.

In a first condition (the punching condition) as seen in FIG. 5 the sample collector driver 626 is engaged with the punch 604 at an interface 671 so that a force applied by the actuator mechanism of the sample device 1300 via the push rod 662 pushing onto the sample collector driver 626 can push the punch 604. The punch 604 and the sample collector driver 626 are in a compact position relative each other for the purposes of such pushing to drive the sample collector and the sample collector driver through the ear of an animal. The punch and plunger are in a retracted position relative each other so that a cavity is defined by the cutter and the plunger to allow a sample to be collected and held in the cavity. The plunger and the sample collector driver are in a first position where the plunger is located in the cavity 672 of the sample collector driver. The umbrella of the plunger is in a contracted condition. So in the first condition the:
1. Sample collector driver and punch are in a compact position,
2. Punch and plunger are in a retracted position, and
3. Plunger and sample collector driver are in a first position with the umbrella in the contracted condition.

The sample collector and preferably also the sample collector driver are, in this first condition, able to be driven through the ear of the animal and engaged with the storage container as seen in FIG. 6.

In a second condition, after the cutter has passed through the ear of the animal and a sample is held in the cavity, as seen in FIG. 9, the plunger 609 remains in a retracted position relative to the punch 604. The punch is snugly held by the cap of the storage container and sample collector driver is moved back towards where it came from by the mechanism of the sampler device. The sample collector driver and punch, in the second condition are in an expanded position relative each other. They are preferably telescopically engaged. The punch and plunger remain in a retracted position but the plunger and sample collector driver are in a second position relative each other where the umbrella is in an expanded condition and no longer in the cavity 672.

So in the second condition the:
1. Sample collector driver and punch are in an expanded position,
2. Punch and plunger are in a retracted position, and
3. Plunger and sample collector driver are in a second position with the umbrella in the expanded condition.

In a third condition as seen in FIG. 15 the sample collector driver is pushed back towards the punch and preferably back to or near its earlier compact position. This is achieved by the driving mechanism of the sampler device that will herein after be described. In the third condition, the plunger has moved to an advanced position relative the punch. The sample collector driver and plunger remain in the second position with the umbrella in the expanded condition. The sample collector driver is also now fully seated with the storage container at the cap where it may seal the storage container.

So in the third condition the:
1. Sample collector driver and punch are in or near the compact position,
2. Punch and plunger are in an advanced position, and
3. Plunger and sample collector driver are in a second position with the umbrella in the expanded condition.

The opposite distal end of the plunger includes an umbrella 665 that is in a contracted condition when the punch and the sample collector driver are in the compact position. The umbrella is retained in a contracted condition by virtue of being in a first cavity 672 of the sample collector driver 626. The first cavity 672 is of a nature that will hold the umbrella 665 of the plunger 609 in a contracted condition as seen in FIG. 3b. In the preferred form the driving end 666 of the plunger is located against the end wall 673 of the first cavity 672. The plunger 609 extends from the first cavity through a second cavity 674 from the sample collector driver 626. Preferably the first cavity 672 is a bore of a first diameter. The second cavity 674 is preferably a bore of a larger diameter and includes an end wall 675 with which the driving end 666 of the plunger can engage when the plunger has been axially displaced relative the sample collector driver 626 and out of the first cavity 672. Upon a displacement the umbrella 665 which is naturally biased to an expanded condition, is able to expand and upon such expansion is then not able to re-enter the first cavity 672. Instead the driving end 666 is able to contact the end wall 675 of the second cavity 674. When in the axially displaced condition relative to the sample collector driver 626 further driving of the sample collector driver 626 towards the storage container will result in the plunger being displaced relative the punch. This occurs by virtue of the interaction of the driving end 666 with the end wall 675 of the second cavity.

The umbrella 665 is preferably integrally formed as part of the plunger 609. The plunger and its umbrella is preferably of a plastics material and the umbrella is preferably naturally formed in an expanded condition but is of a configuration that allows for it to be collapsed to a contracted condition as seen in FIG. 3b.

As can be seen in FIGS. 5 and 6 when the sample collector and sample collector driver are being driven into the ear of the animal the sample collector and sample collector driver are in a compact position and a driving of the punch occurs by the interface between the sample collector driver and the punch. In FIG. 6 it can be seen that a sample 612 has been removed and is held by the cutter 605. The plunger is still in a retracted position relative to the cutter and has not been moved to eject the sample from the cutter 605.

The storage container 602 and preferably its cap 619 is able to snugly receive the punch 604 and be guided for a sliding movement into the bore of the cap 619. The assembly of the sample collector and sample collector driver is able to be driven through the ear of the animal as seen in FIG. 7. As previously described, the axial relationship between the punch and the sample collector driver and the plunger remains the same at this stage of sampling as before the sample was taken. The assembly of the sample collector driver and the sample collector is merely displaced through the ear of the animal and has engaged with the cap of the storage container 2. The sample collector is able to advance in the bore of the cap in order to reach the flangible seal. This seal is preferably of a configuration as hereinbefore described. The punch can push onto, to remove the flangible seal 624.

Preferably once the frangible seal 624 has been broken (but alternatively prior to this point) the axial relationship between the sample collector driver 626 and the sample collector 601 is changed. The sample collector driver 626 is retracted at least partially back towards where it came from and moves at least partially back out of the cap as seen in FIG. 9. The mechanism of the sampling device may cause this displacement. The sample collector 601 (the punch 604 and plunger 609) remain in its position relative to the storage container where it was delivered by the sampler device and does not retract back with the sample collector driver 626. The partial retraction of the sample collector driver 626 causes a relative axial displacement of a distance sufficient to allow for the driving end 666 of the plunger 609 to displace out of the first cavity 672 of the sample collector driver 626. This causes the umbrella to expand. Preferably the retraction distance is substantially no greater than that required for the driving end 666 to move out of the first cavity 672 and allow for the umbrella to expand. The umbrella in its expanded condition is now able to engage the end wall 675 of the second cavity 674 of the sample collector driver 626.

As seen in FIGS. 10 and 11a, the size or shape di of the driving end 666 of the plunger is approximately the same as the size d₂ of the cavity 672 of the sample collector driver. This is when the punch is in its compact position relative to the sample collector driver and the plunger is the first position relative to the sample collector driver. In the second position of the plunger relative to the sample collector driver, the size or shape D₁ of the driving end corresponds to the size or shape D₂ of the second cavity of the sample collector driver.

Further displacement of the sample collector driver 626 into the cap causes the sample collector driver 626 to drive the plunger 609 and thereby will eject the sample 612 from the cutter 605 as seen in FIG. 12.

A continued advancement of the sample collector driver 626 as can be seen in FIG. 15 will cause the sample collector driver 626 to lodge into the passage of the cap and thereby provide an additional seal to the containment region. The sample collector driver 626 may include an enlargement 676 that is able to push into an annular recess 677 of the bore of the cap. The interaction of the enlargement and the annular recess can provide further barrier to the ingress or egress of contaminants and/or sample material to and from the storage region 616 of the storage container 602. The interaction of the enlargement and the annular recess will also help hold the sample collector driver in a fixed relationship to the cap so that a retraction force applied to the push rod 662 will cause the release of the push rod 662 from the sample collector driver 626 as can be seen in FIG. 16.

As can be seen in the sequence of images of FIG. 17-19 in a first condition as seen in FIG. 17 the sample collector driver 626 and punch 604 are in a compact relationship relative to each other and the plunger 609 is in a retracted position relative to the punch 604. This is the punch mode of the assembly. In FIG. 18, the punch and sample collector driver are in their expanded position relative to each other, where the sample collector driver 626 has been retracted away from the punch 604 to allow for the umbrella 665 to expand and for the driving end 666 to contact the end wall 675 of the sample collector driver. An advancement of the sample collector driver 626 back towards the punch 604 will then cause the plunger 609 to displace relative to the punch 604 to its advanced position and for the sample to be ejected. The displacement back towards the punch 604 will also displace the sample collector driver 626 further into the cap than it was when it was in a punch condition as seen in FIG. 17 so that the sample collector driver 626 can fully seal the bore of the cap.

In alternative forms the sample collector driver may be removed from the sample collector after it has driven the sample collector into the storage container and has actuated the plunger. The sample collector driver may not seal the storage container. The sample collector driver maybe disposed.

FIGS. 11b-j show variations to the telescopic ratchet relationship of the plunger and the sample collector driver. FIGS. 11c, e, g and I shows the driver and plunger in where the relationship between the two, when assembled would be where the plunger is in the retracted position and figures d,f,h,j respectively shows the driver and plunger in where the relationship between the two, when assembled would be where the plunger is in the advanced position.

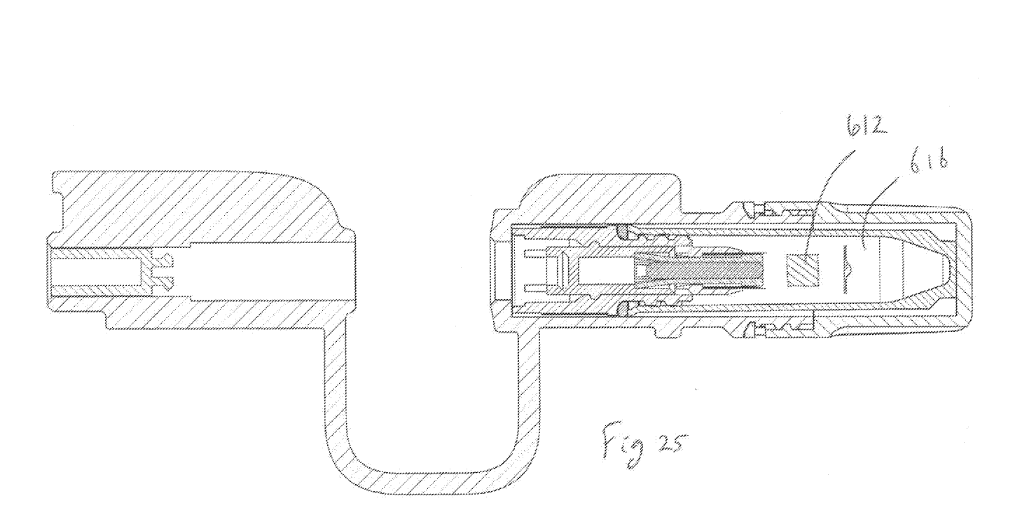

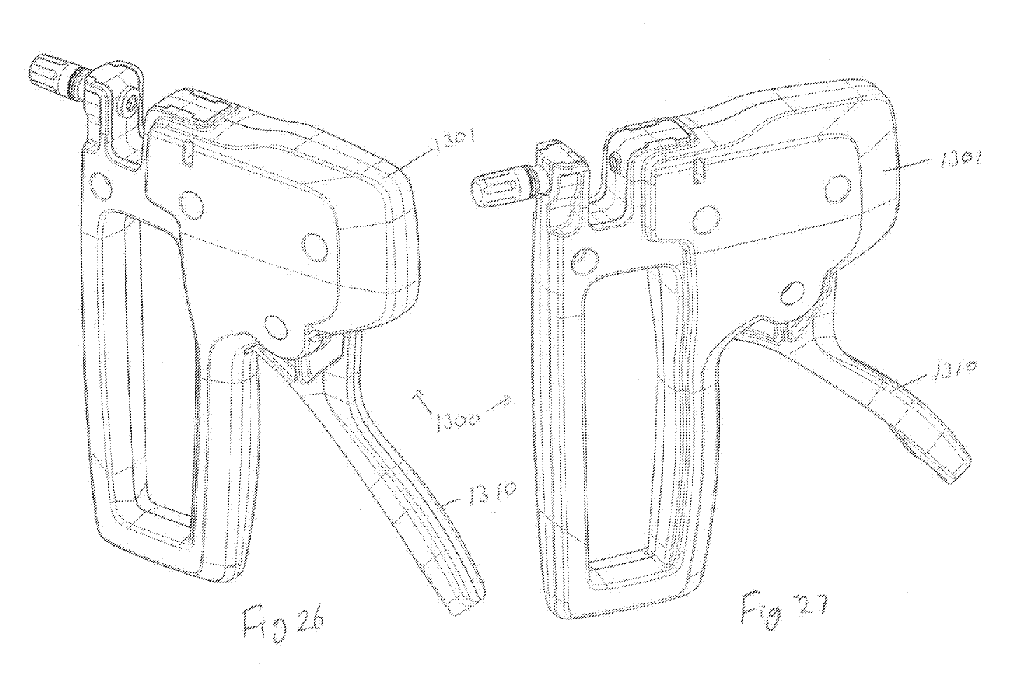

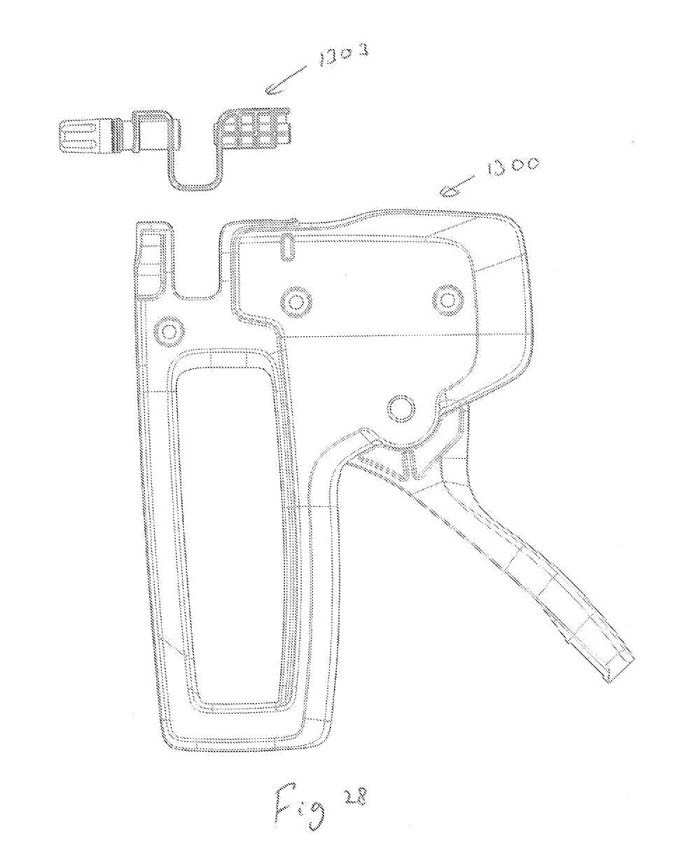

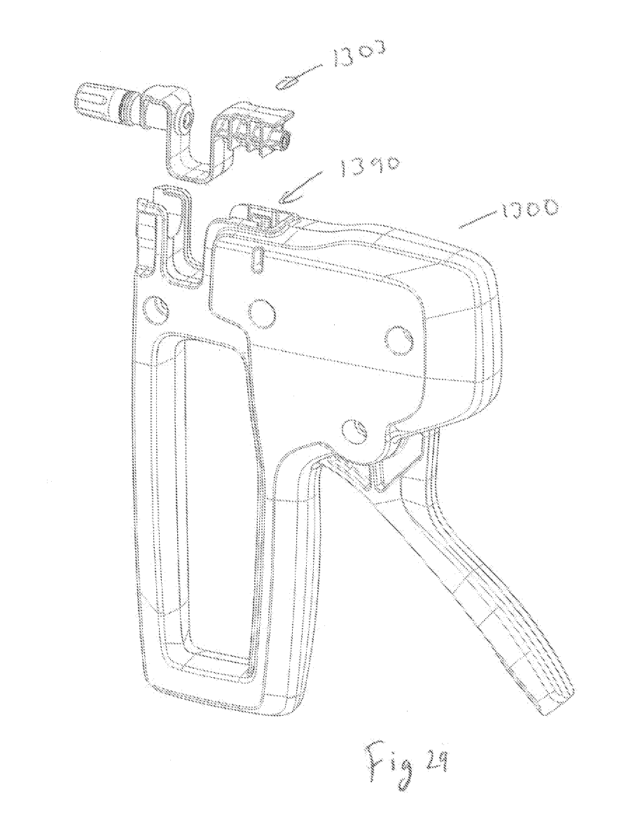

The sample collector and sample collector driver and the storage container may be provided in a form associated with a cartridge 1303. The cartridge may be a body that defines a storage container holding region 1306 and a sample collector and sample collector driver holding region 1307 as seen in FIGS. 20 and 21. An exploded view of this is shown in FIG. 22. The body of the cartridge 1303 holds the sample collector and storage container and sample collector driver in an axially aligned condition. It holds the storage container in a condition separated by a gap 1308 from the sample collector. As seen in FIG. 24, the gap 1308 is sufficiently large to allow for an ear 630 of an animal to be inserted into the gap and be presented to be axially aligned for the purposes of taking the sample. The cartridge 1303 is able to be loaded into a sampler device 1300 as shown in FIG. 26-30. It is preferably able to be removably loaded so that once the sample has been taken, the cartridge can be removed. Upon removal the storage container may remain associated with the cartridge and be shipped as a unit to a laboratory for further processing.

A cross-sectional view of the arrangement of FIG. 21 is shown in FIG. 23. This is the pre-sampling condition where the sample collector driver and sample collector 601 are on one side of the gap 1308 and the storage container 602 is on the opposite side of the gap. FIG. 24 shows the sample collector 601 and sample collector driver 626 assembly being delivered for taking a sample of the ear 603 of an animal. FIG. 25 shows the sample collector and sample collector driver having been delivered to the sample collector, the sample collector driver 626 sealing the cap of the sample collector and the plunger having been actuated to push the sample 612 into the storage region 616.

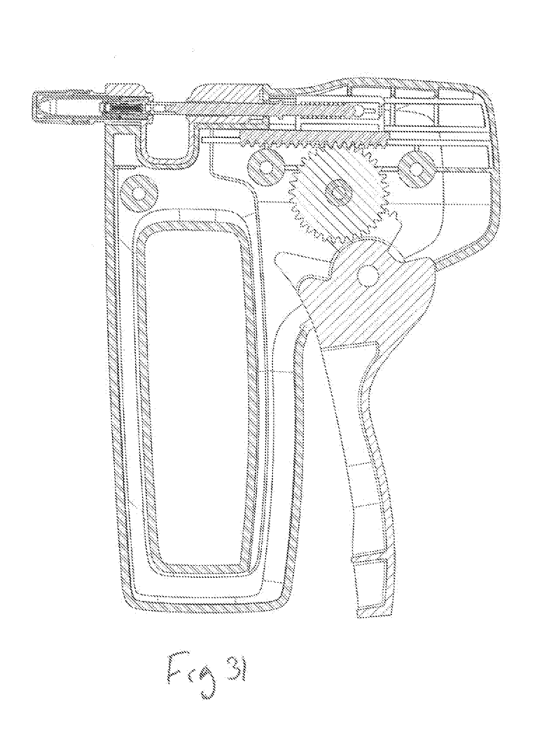

With reference to FIG. 26 there is shown a sampler device 1300 that includes a body 1301 and a handle 1310. A handle is able to be actuated by a user to cause a shaft 1360 of the sampler device to actuate the sample collector and sample collector driver assembly, preferably via the push rod 662, to collect the sample and deliver it to the storage container. The cartridge 1303 is able to be removably associated with the sampler device 1300. In a removed condition shown in FIG. 28 the cartridge 1303 with the sample collector, sample collector driver and storage container pre-associated therewith, (preferably at the factory and prior to delivery of the end point of use), is able to be inserted into the sampler device 1300. The cartridge 1303 is able to lodge and thereat be releasably held.

The cartridge may include an area where a person is able to write information onto. A pen may be used for this purpose. The information may for example be a date and/or an animal number as may be found on an eartag of an animal.

The cartridge may carry an EID. This may carry information that is matched to the EID information of the sample collector and/or the container.

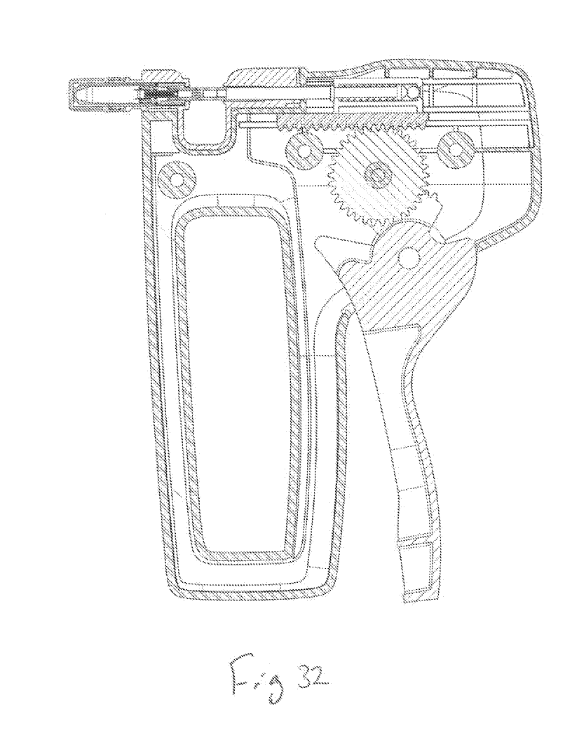

With reference to FIG. 30, the mechanism of the sampler device is shown. The mechanism includes a drive shaft 1360 that is able to be actuated for driving towards the gap 1308, aligned with each of the sample collector storage container, sample collector driver and the push rod when used. All these elements are in alignment and in alignment to the direction of displacement of the drive shaft. The drive shaft is able to move in the direction to deliver the sample collector and sample collector driver across the gap, through the ear of an animal and into the storage container.

The trigger mechanism that is used for the sampler device may be one that causes the movement of the drive shaft in a direction to drive the collector towards the storage container and may manually cause the shaft to be partially retracted in order to partially withdraw the plunger from the sample collector driver so that the umbrella is able to move from its contracted condition to its expanded condition. The drive shaft may then be manually caused to drive back in the driving direction to finalise the movement of the plunger and sample collector driver to the third condition.

This may be caused by moving the handle of the sampler device appropriately both in one direction and in an opposite direction. Alternatively this may be caused by the operation of an intermittent drive mechanism capable of causing the driving, partial retraction, then return to driving under a single driving input. The driving input may be the movement of the handle of the sampler, or could be the operation of a motor such as an electric motor.

Further alternatively the trigger mechanism may be such that the drive shaft is able to be driven in this manner by the action of an adequately controlled motor, such as an electric motor or even more particularly a DC servo motor. An electric motor may be used with or without an intervening intermittent mechanism to drive the drive shaft 1360. Such a configuration is shown in FIG. 34, where a servo motor 1370 drives the drive shaft 1360.

In FIG. 34 the linkage between the servo motor 1371 and drive shaft 1360 may comprise a mechanism wherein the driving rod is allowed to retract, such as under a bias, a certain amount once it has reached a certain point of its extension. The mechanism may then re-engage the linkage to the servo motor 1370, allowing the drive rod 1360 to be driven forwards again.

Alternatively, the linkage between the servo motor 1370 and driving rod 1360 in FIG. 34 may comprised a fixed linkage, without an intermittent mechanism, wherein the extension, retraction and return to extension of the drive rod 1360 are provided by the driving of the servo motor 1370. The control over the timing, speeds and duration of the operation of the drive rod 1360 in each direction may be provided by control of the servo motor 1370. It is contemplated that the use of commonly available control systems and electric motors may in combination be suitable to provide the nature of control, including both speed, direction and location, as desirable to perform the previously described function of the drive rod.

The servo motor 1370 may be powered by an internal power source such as a battery 1371. The operation of the servo motor 1370 may be initialised by an actuation of the handle 1302 resulting in the engagement of a switch 1372. Further alternatively the drive rod 1360 may be coupled to a linear motor, wherein the linear motor is capable of being controlled and operated to drive the drive rod 1360 in both direction as previously described. In such a configuration no drive linkage between the motor and drive rod may be required.

1. As an assembled elongate string sample collector and sample collector driver to drive at least the sample collector through an item to be sampled along a sampling axis aligned to the elongate direction of the string, the sample collector comprising:
a punch having a bore aligned to the elongate direction and presenting at one end of the punch and bore a cutter to cut and hold a sample from the item at the bore as the punch passes through the sample,
a plunger slideably supported to said punch to be able to move in said bore between a retracted position and an advanced position relative said punch, the movement from the retracted position to the advanced position causing, in use, the cutter held sample to be displaced from the cutter,
the sample collector driver comprising
a body interfaced with said sample collector in a telescopic manner wherein in a telescopically compact position the driver can drive the sample collector through the item whilst keeping the plunger in the retracted position so that a sample can be cut and held by the cutter and in a telescopically expanded position the driver is able to be driven in the same direction as when driving the collector through the item and cause the plunger to move from its retracted position to its advanced position.

2. The string as claimed in claim 1 wherein when the sample collector and the body are in the telescopically compact position the body and the punch are in contact with each other so that the body, when being driven, pushes onto the punch to drive the sample collector through the item.

3. The string as claimed in claim 1 wherein when the sample collector and the body are in the telescopically compact position the body and the punch are in contact with each other so that the body, when being driven, pushes onto the punch and not onto the plunger, to drive the sample collector through the item.

4. The string as claimed in claim 1 wherein when the sample collector and the body are in the telescopically compact position the body and the punch are in contact with each other so that the body, when being driven, pushes onto the punch and does not cause the plunger to move to its advanced position, to drive the sample collector through the item.

5. The string as claimed in claim 1 wherein when the sample collector and the body are in the telescopically expanded position the body and the plunger are in contact with each other so that the body, when being driven in the same direction, pushes onto the plunger to move the plunger to its advanced position.

6. The string as claimed in claim 1 wherein when the sample collector and the body are in the telescopically expanded position the body and the plunger are in contact with each other so that the body, when being driven in the same direction, pushes onto the plunger and not onto the punch, to move the plunger to its advanced position.

7. The string as claimed in claim 1 wherein when the sample collector and the body are in the telescopically expanded position the body and the plunger are in contact with each other so that the body, when being driven in the same direction, pushes onto the plunger and does not cause the punch to move, to move the plunger to its advanced position.

8. The string as claimed in claim 1 wherein one of the body and the plunger carries a dog that in said compact position is help in an inactive mode preventing the driving of the plunger by the body to its advanced position and in the expanded position couples said plunger and body.

9. The string as claimed in claim 8 wherein the dog is a resiliently flexible member of one of said body and plunger, held under bias when in its inactive mode by the other of said plunger and body when the sample collector and the body are in the compact position.

10. The string as claimed in claim 8 wherein the dog is a resiliently flexible member of one of said body and plunger, biased towards a position that, when the sample collector and the body are in the expanded position, presents itself for engagement with a driving surface of the other of said plunger and body so that when said body is moved in the same direction, the plunger is driven towards its advanced position.

11-15. (canceled)

16. The string as claimed in claim 8 wherein the plunger includes an umbrella acting as said dog, the umbrella resiliently moveable between a contracted condition and an expanded condition wherein the umbrella is (a) held in one of said contracted and expanded conditions by a lateral surface or surfaces of said body along which said umbrella can slide when the body moves to the expanded position relative the sample collector and (b) can move to the other of the expanded or contracted conditions when the body has moved to the expanded position so that the umbrella can engage said body to allow the body to move the plunger to the advanced position.

17-20. (canceled)

21. The string as claimed in claim 1 wherein the body and the punch form a continuous elongate drivable member when in the compact position.

22. The string as claimed in claim 1 wherein the body includes a receptacle region to receive a driver, directly or indirectly, of a sampler device that can apply a force to cause the sample collector to be driven through the item.

23. The string as claimed in claim 21 wherein the receptacle region is adapted and configured so that it can be engaged by said driver, directly or indirectly, to cause the body to be displaced in a driving direction and in a direction opposite the driving direction.

24. (canceled)

25. As a set a string as claimed in claim 1 and a sample storage container defining a mouth opening leading to a storage region of the storage container, the mouth opening able to receive the string after the cutter has been driven through the item and is carrying a sample.

26. A set as claimed in claim 25 wherein the storage container is adapted and configured to retain the sample collector (preferably the punch) once received at said mouth opening in a manner to allow the driver to be moved relative said sample collector and said storage container in a direction to move it to the expanded position relative the sample collector.

27. A set as claimed in claim 25 wherein said body advances back towards punch as it drives said plunger to its advanced position, the so advancing body causing a sealing of said mouth opening.

28-30. (canceled)

31. A method of taking and storing a sample from an item using the set as claimed in claim 25 by
a. placing the storage container and string on opposed sides of a gap, held by a sampler device in axial alignment, the sampler device including an actuator to act, directly or indirectly on said sample collector driver,
b. placing a part of an item to be sampled in said gap so that part of the item is also in axial alignment,
c. causing the actuator to move in a driving direction to drive said string towards said storage container so that said sample collector is driven into the mouth opening of the storage container so that said punch becomes held by said storage container,
d. causing the actuator to move in a direction opposite said driving direction to allow the sample collector driver to register with said plunger for the purposes of driving said plunger in said driving direction, and
e. causing the actuator to move in the driving direction to cause the sample collector driver to displace the plunger to its advanced position.

32-35. (canceled)

36. A sampling assembly to be driven in one direction relative to and to collect and dispense a biological sample from an item and into a storage container, said assembly comprising:
a) a sample collector comprising:
a punch presenting at one end a cutter to cut and hold a sample from the item as it passes there through or over,
a plunger slideably supported by said punch to be able to move between a retracted position and an advanced position relative said punch, movement from the retracted position to the advanced position causing, in use, the cutter held sample to be displaced from the cutter,
b) a sample collector driver comprising:
a body telescopically interfaced with said sample collector to, in a first position relative said punch and plunger, be able to drive said punch and plunger in said one direction, and in a second position relative said punch and plunger, having moved in a direction opposite said one direction, having ratcheted to engage with said plunger in a manner to be able to cause said plunger to move relative to said punch when said sample collectors driver is again driven in said one direction.

37. A sampling string to collect a biological sample from an item, said string comprising:
a) a sample collector comprising:
a punch presenting at one end a cutter to cut and hold a sample from the item as it passes there through or over,
a plunger slideably supported by said punch to be able to move between a retracted position and an advanced position relative said punch, movement from the retracted position to the advanced position causing, in use, the cutter held sample to be displaced from the cutter,
b) a sample collector driver that in a first condition is engaged to said punch to, in use, be able to drive said punch over or through said item when in a first position relative punch and is able to telescopically slide relative said plunger to a second condition that is away from and/or not engaged to said punch to ratchet with said plunger and engage therewith to be able to drive the plunger from its retracted position to its advanced position.

38-48. (canceled)
